(19) United States
(12) Patent Application Publication
(10) Pub. No.: US 2007/0293479 A1
(43) Pub. Date: Dec. 20, 2007

(54) OLANZAPINE PHARMACEUTICAL COMPOSITION

(76) Inventors: Niels J. Osinga, Nijmegen (NL); Farid Abedin Dorkoosh, Utrecht (NL)

Correspondence Address: SYNTHON IP INC, 7130 HERITAGE VILLAGE PLAZA, STE 202, GAINESVILLE, VA, 20155, US

(21) Appl. No.: 11/750,730

(22) Filed: May 18, 2007

Related U.S. Application Data
(60) Provisional application No. 60/747,624, filed on May 18, 2006.

(51) Int. Cl. A61K 31/551; A61P 25/18

(52) U.S. Cl. 514/220

(57) ABSTRACT

An olanzapine pharmaceutical composition is formed using anhydrous calcium hydrogen phosphate. The composition can be tabletted by dry processes and typically has good stability.

This application claims the benefit of priority under 35 U.S.C. § 119(e) from prior U.S. provisional patent application No. 60/747,624, filed May 18, 2006, the entire contents of which are incorporated herein by reference.

### BACKGROUND OF THE INVENTION

The present invention relates to a solid pharmaceutical composition comprising olanzapine as the active ingredient.

Olanzapine is represented by the structural formula (1) 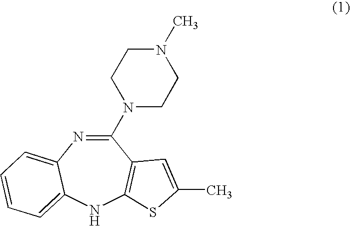
and is a pharmaceutically useful compound. In medical treatments, it is useful as an antipsychotic agent, particularly for the treatment of schizophrenia. The marketed final forms include coated tablets and quick dissolvable tablets. The single tablet comprises from 2.5 to 20 mg of olanzapine.

In the present commercially available final forms the active substance is marketed as a free base. It is a white to yellow crystalline solid that is insoluble in water (solubility at pH 6.8 is about 0.02 mg/ml).

Olanzapine and pharmaceutically acceptable salts have been suggested in EP 454436 and corresponding U.S. Pat. No. 5,229,382. In the final stage of the production process, olanzapine was obtained by a crystallization of the crude olanzapine product from acetonitrile. The patent does not refer to or identify any specific crystalline form of olanzapine.

Later, it became known that olanzapine base may exist in various crystalline modifications and in hydrated/solvated forms that are stable at ambient conditions (For example, see EP 733635/U.S. Pat. No. 5,736,541, WO 98-11893, and EP 831098).

The term “Form I olanzapine” was later designated in EP 733635 to the anhydrous olanzapine product that was stated to be obtainable according to the process of U.S. Pat. No. 5,229,382.

EP 733635/U.S. Pat. No. 5,736,541 discloses Form II olanzapine which is characterized by a main X-ray powder diffraction peak of d-value 10.26 A. This form has been prepared by crystallizing “technical grade” olanzapine (the product of the earlier synthesis) from ethyl acetate. This form appears to be more stable than the Form I, but it is convertible to Form I. Similarly as Form I, the Form II is an anhydrate.

U.S. Pat. No. 6,348,458 (WO 01/47933) discloses additional crystalline polymorphic forms of olanzapine, namely Form III, Form IV and Form V. These forms are made by neutralizing an acid solution of olanzapine by the addition of alkali under varying conditions to precipitate the desired olanzapine crystalline Form.

More recently, WO 03/091260 discloses Form VI olanzapine and U.S. Appl. Publication No. 2002-0086993 discloses a polymorphic form designated as form X.

As the system used for numbering the known olanzapine forms is sometimes confusing in the prior art disclosures (for instance, the EP 828494 calls as olanzapine Form I a product that is identical with olanzapine Form II of the above definition), the “Form I” of olanzapine as used herein is defined as the solid state form of anhydrous olanzapine base which is characterized by a main peak on the X-ray powder diffraction spectrum of d-value 9.9463 A. The full diffraction pattern of the Form I has been disclosed in EP 733635. The “Form II” of olanzapine as used herein has the same definition as used in EP 733635/U.S. Pat. No. 5,736,541, namely it is characterized by a main X-ray powder diffraction peak of d-value 10.26 A.

Interestingly, WO 02/18390 indicates that upon repetition of the disclosed process in U.S. Pat. No. 5,229,382, the product obtained does not correspond to the Form I. Instead a Form II olanzapine is obtained after the crystallization from acetonitrile, while a hydrated olanzapine is obtained prior to the crystallization. The Form I complying with the above definition was actually prepared in WO 02/18390 by recrystallization of olanzapine Form II or a hydrate of olanzapine from dichloromethane, followed by drying of the wet product at 60-70° C. In fact, the product of crystallization is a dichloromethane solvate of olanzapine, which liberates dichloromethane under the conditions of drying and yields the Form I.

The original olanzapine patent, i.e., EP 0454436B1 and U.S. Pat. No. 5,229,382, describe that various pharmaceutical compositions of olanzapine can be prepared. A specific example of a tablet is provided which contains starch, microcrystalline cellulose, povidone, and magnesium stearate as excipients.

However, EP 0733367 B1/U.S. Pat. No. 5,919,485 indicate that the known olanzapine tablets had the tendency to discolor, which can be especially problematic to a psychotic patient. Apparently, olanzapine is metastable and moisture sensitive. Further, it undergoes discoloration when contacted with certain (unspecified) excipients including powder blends. The discoloration is enhanced by ambient air, elevated temperature, and/or moist environments. And because of the high potency of olanzapine, there were apparent concerns about assuring homogeneity of the finished solid formulation. The purported solution to these issues was the inclusion of a polymeric coating around the solid oral formulation (e.g. a tablet). This coating provides improved resistance to discoloration. Preferably the coating was used as a sub-coat with a white color coating thereover. The tablet itself preferably contains lactose as the main diluent or “bulking agent.” The tablets are preferably made by aqueous wet granulation with fluid bed drying because direct compression and dry granulation had a greater chance of poor dose uniformity.

EP 0830858 A1 and published U.S. application 2001/0020032 also relate to solving the discoloration problem of olanzapine tablets. Instead of the tablet being coated, however, here the olanzapine powder is coated with a polymer to protect it from discoloration. The technique is especially useful in making granules which can be compressed into tablets. The examples use wet granulation.

WO 2004/035027 recites an olanzapine pharmaceutical composition comprising a homogeneous mixture of (a) olanzapine or a pharmaceutically acceptable salt thereof as an active ingredient, (b) a monosaccharide and/or oligosaccharide, and (c) a polysaccharide. The composition can additionally contain a binder, a disintegrant, and a lubricant. Preferably the composition is an uncoated tablet which is preferably prepared by direct compression. The tablets are reported as being stable and not suffering from discoloration. According to this publication, the discoloration is caused by the conversion of olanzapine into olanzapine hydrates but this could be prevented by homogeneously mixing the olanzapine with certain excipients and then performing direct compression. The dose uniformity is stated to be excellent despite the use of direct compression.

WO 2005/0009407 discloses olanzapine pharmaceutical compositions that are also supposed to be stable against discoloration. The proposal involves coating olanzapine particles or powder with a coating that includes lactose and/or mannitol and optionally other excipients. The coated olanzapine particles can be formulated into granules or tablets. The examples use a number of excipients (seven or eight including typically two kinds of microcrystalline cellulose) and steps (a coating step and wet granulation step, etc.) before obtaining granules ready for tabletting or filling into capsules.

It is desirable to provide a stable pharmaceutical composition comprising olanzapine, particularly the Form I of olanzapine. It is also desirable to form an elegant pharmaceutical tablet by a relatively simple process, such as one that does not use solvent or water. Further, it is desirable to form an olanzapine tablet that does not need a special coating to remain stable and that preferably avoids significant discoloration upon storage.

### SUMMARY OF THE INVENTION

The present invention relates to the discovery of a simple, easy to produce olanzapine pharmaceutical composition that has good stability by the use of anhydrous calcium hydrogen phosphate as the main excipient. Accordingly, a first aspect of the invention relates to a pharmaceutical composition comprising 1-10% olanzapine, preferably Form I olanzapine; 50-85% of an anhydrous calcium hydrogen phosphate; and 10-30% of a binder, preferably microcrystalline cellulose. Other excipients can also be present especially a disintegrant and a lubricant.

In particular, the calcium hydrogen phosphate is a coarse grade calcium hydrogen phosphate with an average particle size between 50-100 microns. Additionally the calcium hydrogen phosphate preferably has a pH of between 6.5 to 7.5 when measured in a 20% aqueous slurry.

The composition can be formed into tablets containing 1-50 mg of olanzapine per tablet. Preferably composition and the tablets are made by a process of mixing the components in which a liquid is absent, i.e., a dry process such as direct compression.

Surprisingly the above described composition can be formed into tablets having good stability, with reduced discoloration, and good content uniformity.

### DETAILED DESCRIPTION OF THE INVENTION

Within the present invention, the word “olanzapine” means the anhydrate form of olanzapine base and does not include olanzapine hydrate(s) or olanzapine solvate(s).

During the thorough research on tablettable pharmaceutical compositions comprising olanzapine as the active ingredient, and particularly the solid state Form I thereof, it was found out that many compositions suffer from a progressive formation of an undesirable impurity, which has been identified as a lactam compound 2-Methyl-5,10-dihydro-4H-thieno[2,3-b][1,5]benzodiazepin-4-one of the formula (II)

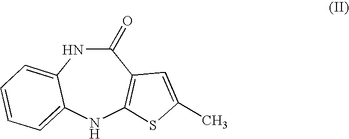

In attempts to reduce the amounts of the lactam impurity formed during prolonged storage of olanzapine compositions, it appears that the degree of the lactam formation is dependent on the presence of water in the composition and also on pH, e.g., the micro environmental pH of the composition. Surprisingly, it was observed that its formation may be suppressed in compositions where large amounts of anhydrous calcium hydrogen phosphate are present. Furthermore, it was found out that such compositions have a low tendency to change color, and can have a very good content uniformity even when no liquid has been used within the homogenization process. Thus, the composition can be economical from a variety of aspects, including the use of convenient direct compression tabletting techniques.

Calcium hydrogen phosphate anhydrous (CaHPO4, also known as dibasic calcium phosphate anhydrous) is a well known pharmaceutical excipient, particularly for making tablets. It is commercially available in two grades, milled grade and coarse grade. Both grades are equally useful within the present application, but the coarse grade is preferred due to its better applicability in the direct compression tabletting technique, which is considered as the most preferred due to its simplicity. The coarse grade calcium phosphate has conventionally an average particle diameter between 50-100 microns. Brands of anhydrous calcium hydrogen phosphate available for direct compression include A-TAB (Rhodia), Di-Cafos A (Chemische Fabrik Budenheim), Emcompress (Penwest Pharmaceuticals Co.), and Fujicalin (Fuji Chemical Industry Co. Ltd.): Di-Cafos A is preferred.

Calcium hydrogen phosphate is insoluble in water, but it still contains some amount of ions so that a pH of an aqueous slurry is measurable. Based on the source, the pH may vary from 5 to 7.5 or more (in a 20% aqueous slurry). The surface of milled anhydrous calcium hydrogen phosphate may be alkaline and, if so, can be problematic with drugs that are sensitive to alkaline pH. However, there are differences in the surface alkalinity/acidity between the milled and unmilled grades of anhydrous dibasic calcium phosphate; the unmilled form generally having a more acidic surface environment. This difference may have important implications for drug stability, particularly in a process in which the particle size of the anhydrous calcium hydrogen phosphate might be expected to change.

As it appears that the formation of the lactam impurity is pH-dependent, wherein its formation is the lowest at the pH around 7 and is pronounced particularly in the acidic pH, a calcium hydrogen phosphate having an environmental pH of between 6.5 to 7.5 is preferred in the compositions of the invention. It is expected that the overall pH of the composition or tablet may also have an effect on stability and thus relatively neutral to alkaline pH are likely to be preferable.

The anhydrous CaHPO4 contains only a negligible amount of water (e.g., surface-adsorbed moisture) and cannot be re-hydrated to form a hydrated form. This is an advantageous aspect, as the reported color change of olanzapine is apparently associated with formation of olanzapine hydrates. Formation of hydrates consequently could have an impact on bioavailability, e.g., the solubility properties of olanzapine hydrates can differ from those of the anhydrate material. The formation of hydrates may be induced also by the presence of water in other excipients, such as binders, disintegrants etc., and may also be induced by environmental moisture. Surprisingly enough, the presence of anhydrous calcium hydrogen phosphate in the composition reduced the need of using solely anhydrous excipients in the composition. Therefore, excipients such as microcrystalline cellulose, which in commercial grades inherently contains several percent of water, may nonetheless be used within the compositions of the present invention. Also a protective coating against the environmental humidity is generally not necessary.

Tabletting processes, in which water is employed, such as wet granulation, can be avoided in making dosage forms from the composition of the invention. Furthermore, wet granulation processes using a nonaqueous solvent, such as ethanol, should preferably be avoided as a solvent induced solid-solid polymorphic transition may occur, particularly when using the Form I olanzapine.

The relative content of olanzapine in the compositions of the present invention, expressed in respect to the mass of the overall composition, is between 1 and 10%, typically 2-8% (all percents used herein refer to weight percent unless otherwise specified).

The preferred solid state form of olanzapine is the Form I olanzapine, but other polymorphic Forms of anhydrous or otherwise non-solvated olanzapine are equally useful. Form I olanzapine generally was found to have good tabletting properties. It is even well compatible with calcium hydrogen phosphate, typically not exhibiting significant segregation therefrom, even in the high calcium hydrogen phosphate content used in the present invention.

The olanzapine used in the present invention can be made by any suitable synthesis technique such as those described in the above-mentioned prior art. Similarly, the Form I olanzapine can be made by any suitable method such as the known processes/techniques. More recent suitable processes for making Form I olanzapine include those described in published U.S. applications 2007/0066602; 2007/0021605; and 2005/0272720.

The anhydrous calcium hydrogen phosphate comprises at least half of the composition of the present invention, generally 50-85%, typically 60-75%, and in some embodiments 65-75%. In light of the fact that anhydrous calcium hydrogen phosphate is a very cheap excipient, the possibility that a large amount thereof may be present in the composition makes the composition economically advantageous.

As stated above, the calcium hydrogen phosphate shall preferably exhibit a pH of between 6.5 to 7.5 to suppress the formation of the lactam impurity during the manufacturing process and prolonged storage. The pH value may be measured by slurrying the calcium hydrogen phosphate in water (20% aqueous slurry).

The coarse grade calcium hydrogen phosphate anhydrous is preferred for its better tabletting properties than the milled grade. This is specifically important for the process of direct compression, i.e. in a tabletting process without a prior granulation. As pointed out above, generally expectable problems of the content uniformity and compatibility of the drug with calcium hydrogen phosphate are surprisingly not problematic in the compositions of the present invention.

From all these aspects, the Di-Cafos A coarse grade of calcium hydrogen phosphate anhydrous is preferred within this invention. It is characterized by the producer by a high bulk density (±1300 g/l), and a pH of 7.0 in a 10% slurry (actually 7.2 in 20% slurry).

In a dry granulation process, e.g. in a process using a roller compaction, the milled grade calcium phosphate may be used as well.

The composition of the present invention typically further contains a binder, especially a water insoluble binder suitable to aid in direct compression. Generally the binder is microcrystalline cellulose, which can also aid in obtaining good content uniformity of the tablets. Other suitable binders include methyl cellulose and starch. The binder comprises less than half of the weight of the composition and is generally 10-30% of the composition. In some embodiments the binder amounts to around 20%; e.g. 15-25%; 17-23%; or even 18-22%, of the total mass of the tablet composition.

Although small amounts of water present in the microcrystalline cellulose binder do not contribute to the formation of impurities or in color changes of the composition, it is nonetheless generally preferred that the presence of water in these and any other excipient should be limited.

Generally the composition of the present invention also contains a disintegrant. The disintegrant is generally used only in small amounts and serves as a tool for better disintegration of the tablet composition in the stomach after oral administration. A typical disintegrant is sodium starch glycolate.

Other conventional excipients may also be present in the compositions of the present invention. For example, a glidant such as talc, colloidal silicon dioxide, etc. and/or a lubricant such as magnesium stearate, calcium stearate, glyceryl behenate etc, to improve the flowability of the composition during tabletting process and to avoid stickiness to tablet punches can be included.

The composition of the present invention is preferably made by a direct mixing of the components, without using a homogenization process (or a granulation process) in which a homogenization/granulation liquid is used; e.g., a “dry process.” To reach the desired content uniformity, the olanzapine is preferably first mixed with one excipient, e.g. with (at least a part of) the anhydrous calcium hydrogen phosphate and/or with the binder and thoroughly homogenized, e.g. in a high-sheer mixer. Afterwards, the remaining excipients are added and homogenized again. Finally, the lubricant is added to the homogenized composition, as conventional in the art. The composition is then ready for tabletting.

The final dosage form made from the composition of the present invention is a tablet. The tablet comprises a therapeutically suitable amount of olanzapine within the above composition. The suitable mass of the tablet is from 100 to 400 mg, advantageously from 150 to 350 mg. The tablets preferably exhibit a hardness from about 40 to about 130 N. The amount of the olanzapine drug in a tablet is preferably from 1 to 50 mg.

Protective coating of the tablets is, in essence, not necessary. If desired, a tablet may be film-coated to improve its appearance and handling using conventional film-coating materials and techniques.

The tablets may be used in treatment of olanzapine-treatable diseases in dosages and regimens similar to the marketed olanzapine tablets.

### EXAMPLES

### Example 1

Olanzapine Base Form I—Composition for a Tablet with 5 mg Olanzapine

Unit mass

| Excipient | (mg) | (%) |
|---|---|---|
| Olanzapine base Form I | 5.0 | 2.5 |
| Di-Cafos A | 143 | 71.5 |
| Sodium starch glycolate (Explotab) | 10.0 | 5.0 |
| MCC PH-102 | 40 | 20 |
| Mg stearate | 2.0 | 1.0 |

Batch was prepared in the following way:

A preblend was obtained by sieving olanzapine and part of the anhydrous calcium hydrogen phosphate Di-Cafos A (1:3 ratio) through a 600 um sieve, and blending them in a Turbula mixer for 20 min at 46 RPM.

(B) The remaining Di-Cafos A, the microcrystalline cellulose (MCC), and sodium starch glycolate were sieved through 600 um sieve, added to the preblend, and blended in the Turbula mixer for 20 min at 46 RPM.

(C) Magnesium stearate was sieved through 600 um sieve, added to the blend, and blended in the Turbula mixer for 3 min at 46 RPM.

(D) Tablets were compressed on the Korsch EK-0 excenterpress with round 8 mm punch.

### Example 2

Olanzapine Base Form I—Composition for a Tablet with 20 mg Olanzapine

Unit mass

| Excipient | (mg) | (%) |
|---|---|---|
| Olanzapine base Form I | 20.0 | 6.25 |
| Di-Cafos A | 216.8 | 67.75 |
| Sodium starch glycolate (Explotab) | 16 | 5.0 |
| MCC PH-102 | 64 | 20 |
| Mg stearate | 3.2 | 1.0 |
| Total | 320 mg | 100% |

The composition was prepared by the same procedure as in Example 1 but with the difference that tablets were compressed with 11 mm oblong punch.

Each of the patents and patent applications mentioned above are incorporated herein by reference. The invention having been described it will be obvious that the same may be varied in many ways and all such modifications are contemplated as being within the scope of the invention as defined by the following claims.

What is claimed is:

1. A pharmaceutical composition comprising:
(a) 1-10% olanzapine;
(b) 50-85% of an anhydrous calcium hydrogen phosphate; and
(c) 10-30% of a binder.

2. The pharmaceutical composition according to claim 1, wherein said olanzapine is Form I olanzapine.

3. The pharmaceutical composition according to claim 1, wherein said binder is microcrystalline cellulose.

4. The pharmaceutical composition according to claim 1, wherein said anhydrous calcium hydrogen phosphate comprises 60-75% of said composition.

5. The pharmaceutical composition according to claim 1, wherein said anhydrous calcium hydrogen phosphate has an average particle size in the range of 50-100 microns.

6. The pharmaceutical composition according to claim 1, wherein said anhydrous calcium hydrogen phosphate has pH with the range of 6.5-7.5 in a 20% aqueous slurry.

7. The pharmaceutical composition according to claim 1, wherein said anhydrous calcium hydrogen phosphate is Di-Cafos A.

8. The pharmaceutical composition according to claim 1, which further comprises a disintegrant and a lubricant.

9. The pharmaceutical composition according to claim 1, wherein said olanzapine is Form I olanzapine and is contained in an amount from 2-8%, said anhydrous calcium hydrogen phosphate is contained in an amount from 65-75%, and said binder is microcrystalline cellulose and is contained in an amount of 15-25%.

10. The pharmaceutical composition according to claim 1, wherein said composition is a tablet.

11. The pharmaceutical composition according to claim 10, wherein said tablet is not coated with a polymer.

12. The pharmaceutical composition according to claim 10, wherein said olanzapine is Form I olanzapine and is contained in an amount from 2-8%, said anhydrous calcium hydrogen phosphate is contained in an amount from 65-75%, and said binder is microcrystalline cellulose and is contained in an amount of 15-25%.

13. The pharmaceutical composition according to claim 12, wherein said tablet contains 1 to 50 mg of said olanzapine.

14. The pharmaceutical composition according to claim 13, wherein said tablet has weight in the range of 100 to 400 mg.

15. The pharmaceutical composition according to claim 13, which further comprises sodium starch glycolate and magnesium stearate.

16. The pharmaceutical composition according to claim 12, wherein said tablet was made by a dry process.

17. The pharmaceutical composition according to claim 14, wherein said tablet was made by a dry process.
